(19) United States
(12) Patent Application Publication
(10) Pub. No.: US 2005/0048554 A1
(43) Pub. Date: Mar. 3, 2005

(54) Method of making and using hybrid polymeric thin films for bio-microarray applications

(76) Inventors: Jizhong Zhou, Oak Ridge, TN (US); Xichun Zhou, Oak Ridge, TN (US)

Correspondence Address: QUARLES & BRADY LLP, 411 E. WISCONSIN AVENUE, SUITE 2040, MILWAUKEE, WI, 53202-4497, US

(21) Appl. No.: 10/920,608

(22) Filed: Aug. 18, 2004

Related U.S. Application Data
(63) Continuation-in-part of application No. 10/326,031, filed on Dec. 19, 2002.

(51) Int. Cl. C12Q 1/68; C12M 1/34

(52) U.S. Cl. 435/6; 435/287.2

(57) ABSTRACT

Platforms for easy and cost-effective fabrication of bio-microarrays are disclosed. In one embodiment, the platform contains a substrate having a surface coated with a film of alternating polycationic and polyanionic polymers. In another embodiment, the platform contains a substrate having a surface coated with a polyelectrolyte-silica sol-gel film. Also disclosed are bio-microarrays fabricated using the above platforms and methods of making the platforms and the microarrays.

### CROSS-REFERENCE TO RELATED APPLICATIONS

This application is a continuation-in-part application of U.S. patent application Ser. No. 10/326,031, filed on Dec. 19, 2002, which is herein incorporated by reference in its entirety.

### STATEMENT REGARDING FEDERALLY SPONSORED RESEARCH OR DEVELOPMENT

This invention was made with United States government support awarded by the following agency: Department of Energy, Grant Number KP1301010. The United States has certain rights in this invention.

### BACKGROUND OF THE INVENTION

The use of microarray-based technology is growing rapidly and has had considerable impact in genomic and proteomic research [1-3]. One crucial component of microarray technology is the surface chemistry of the substrate. The chemistry should be suitable for spotting and immobilizing a variety of biological active molecules (DNA, proteins and cells) such that their biomolecular interactions may be evaluated. Therefore, strong emphasis is placed on developing innovative chemistries that provide high binding capacity, efficient hybridization, low background, good spot uniformity, and stability.

A variety of surface chemistries have been described for DNA microarray fabrication. These include in situ synthesis of DNA directly on glass substrates by photolithography or inkjet printing technology [3-5], and the immobilization of pre-synthesized DNA to the substrate surface by chemical or physical attachment [6-13]. The chemical attachment requires activation of the substrate surface with cross-linking reagents and modification of DNA probes with reactive groups [6-11]. While the covalent bonding of DNA on the slide surface usually provides good stability and reproducibility, surface derivatization and the use of cross-linker reagents involves the use of toxic chemicals. The modifications of DNA probes with active groups also add considerable expense.

Physical attachment occurs through noncovalent interactions (i.e., hydrophobic interactions, electrostatic interactions, and entrapment in porous structures) between the DNA and the surface coatings of the substrate used for fabrication of DNA microarrays. The use of poly-L-lysine (PLL) and aminosilane coatings are examples of this approach [12-14]. These methods do not require terminal modifications of DNA probes and are easy to handle. However, it has been reported that these methods have low binding capacity that can lead to experimental inconsistencies and inconclusive data interpretation [13].

The thickness of the coating film deposited on the slide substrate is also an important factor for microarray performance. Two-dimensional (2-D) and three-dimensional (3-D) films have been used thus far for microarray fabrication. The 2-D coatings are usually monolayer of organic molecules containing active groups, such as thiol [6], amine [11-14], aldehyde and epoxy [7-9, 15] which bind DNA probes. These 2-D coatings are usually less than 10 nm thick. Thus, long spacer arms of C12, C16 or poly(dT) are necessary in the oligonucleotide probes in order to improve the accessibility of target DNA [7-9, 15]. The DNA microarrays fabricated using 2-D monolayer coatings have the advantages of good reproducibility and low background signal under fluorescent detection, but have the disadvantages of low binding capacity, hybridization efficiency, and narrow dynamic ranges.

The 3-D coatings are usually constructed by depositing thick polymer films on slide supports. The 3-D platforms for microarray fabrication include acrylamide gel pads or gelatin pads structured by photolithography [16, 17], aldehyde activated agarose film [18], hydrogel polymer [19] and nitrocellulose film [20]. The thickness of these 3-D coatings is usually above the micrometers level. The thick polymeric films increase the number of coupling sites by introducing additional reactive groups through branched linker molecules, which can provide higher probe binding capacity, and thus give higher signal intensity and wider dynamic ranges. However, compared to 2-D coatings, the 3-D coatings have lower reproducibility and a higher background signal caused by auto-fluorescence of the polymer materials.

Protein microarrays are useful for a variety of applications, such as identifying protein-protein interactions, enzyme assays, drug screening, tissue and serum protein profiling, and antibody characterization. However, protein-based microarrays face several additional challenges. Proteins are generally attached and analyzed on activated aldehyde slides [27], where the primary amines and amino terminal amines of the proteins can react readily with the aldehydes of the slide to form a covalent bond. However, in general, proteins are more sensitive to their surrounding environment than are nucleic acids. The hydrophobic nature of many glass and plastic surfaces can cause protein denaturation. Thus, substrate choice is a major consideration when designing protein microarray experiments. Ideally, proteins should be immobilized on a slide support in a way that preserves their native format and their folded conformations. To increase binding capacity, porous substrates such as organic hydrogel nitrocellulose film have been used for fabricating protein microarrays. Protein microarrays produced on these slides suffer from high background signal and high cost because special equipment and engineering processes are required to produce an even film of hydrogel and nitrocellulose on slide surfaces.

A microarray platform that is inexpensive and can be flexibly designed to suit special needs is in great demand for the fabrication of high-throughput polynucleotide and polypeptide microarrays.

### SUMMARY OF THE INVENTION

In one aspect, the present invention relates to a platform for fabricating bio-microarrays wherein the platform comprises a solid substrate and a hybrid film coating a surface of the substrate. In one embodiment, the hybrid film has a structure of alternating layers of polycationic and polyanionic polymers and the total number of polymer layers is at least 2, preferably at least 3, and more preferably from 3 to 15, 6 to 12, or 8 to 10. This embodiment of the hybrid film is also referred to as the multilayered polyelectrolyte thin film or PET in the specification. In another embodiment, the hybrid film is a polyelectrolyte-silica sol-gel film. The hybrid films of the present invention are rich in electric charges, 3-D porous structures, and potentially hydrogen bond-forming groups and thus can immobilize biological matters by electrostatic and porous adsorptions and potentially hydrogen bonds. Therefore, no specific modifications on the biological matters and the hybrid films (e.g., chemically modifying the biological matters and the hybrid films with biotin and streptavidin, respectively) are necessary in order for the biological matters to be immobilized on the films.

In another aspect, the present invention relates to a bio-microarray that contains a biological matter immobilized onto the hybrid film of a platform described above. When the microarray is formed on a hybrid film of alternating layers of polycationic and polyanionic polymers, at least two species of polynucleotides or polypeptides that are free of modifications for the purpose of attaching to the film are immobilized on the film directly to form at least two detection elements and the distance between the centers of the two detection elements is 1 mm or less, 0.5 mm or less, 0.3 mm or less, or 0.2 mm or less. For other types of bio-microarrays of the present invention, it is preferred that at least two species of the same type of biological matter (e.g., cellular organelles, cells, and tissue samples) that are not modified for the purpose of attaching to a hybrid film of the present invention are immobilized on the film to form at least two detection elements and the distance between the centers of the two detection elements is 1 mm or less, 0.5 mm or less, 0.3 mm or less, or 0.2 mm or less.

In still another aspect, the present invention relates to a method of making a platform for fabricating bio-microarrays. The method involves providing a substrate suitable for fabricating bio-microarrays and coating a surface of the substrate with a hybrid film described above.

In one embodiment, the surface of the substrate is coated with a hybrid film of alternating layers of polycationic and polyanionic polymers by depositing a layer of a first polyionic polymer on the surface, depositing a layer of a second polyionic polymer on the first layer wherein the charge of the second polymer is the opposite of that of the first polymer, and repeating the above steps until a desirable number of polymer layers are deposited. If the surface of the substrate is not charged or sufficiently charged for attaching the first polyionic polymer layer by electrostatic adsorption, it should be modified to carry sufficient charge opposite to that the first polyionic polymer. In this embodiment, one or both of the following steps are also optionally performed. The first step involves exposing the polymers coating the substrate to a solution having a pH value of about 4.5 to about 9.5, about 6 to about 8, or about 7.5. This can be achieved through either a separate step or in combination with the step of depositing the last polymer layer in a solution with a desired pH value. This pH treatment step can enhance the performance of the bio-microarrays formed on the films, especially polynucleotide and polypeptide microarrays. The second optional step involves exposing the hybrid film coated substrate to an energy source (e.g., heat, ultraviolet light, or microwave) to further stabilize the association between the film and the substrate and the association between adjacent polymer layers. It may also increase the size of the pores in the film for better immobilizing biological matters. The exact amount of energy (e.g., temperature) and treatment duration employed in this step can be readily determined by a skilled artisan for the particular film being treated. An example is to heat the slides at a temperature of at least 50° C., preferably between 60° C. and 200° C., more preferably between 80° C. and 180° C., and most preferably between 100° C. and 120° C.

In another aspect, the present invention relates to a method of making bio-microarrays. The method involves providing a platform as described above and attaching a biological matter to the film of the platform.

The platforms and microarrays made according to the above methods are also within the scope of the present invention.

In another aspect, the present invention relates to a kit that comprises one or more uncoated substrates such as substrates made of glass, silica, or plastic (e.g., nylon) together with vials or containers of predetermined volumes of suitably buffered solutions of polymers (polycationic polymers and polyanionic polymers) suitable for making a hybrid film-coated substrate of the present invention, and instructions for application of the solutions to the substrates to form hybrid film-coated substrates.

In yet another aspect, the invention relates to a kit for fabricating a microarray on a hybrid film-coated substrate (e.g., glass, silica, or plastic) of the present invention. The kit comprises one or more substrates having a hybrid film-coated surface and a suitable solution for immobilizing biomolecules (e.g., polynucleotides and polypeptides) on the hybrid film by electrostatic adsorption and entrapment of the porous structures within the hybrid film. Optionally, an instruction for immobilizing the biomolecules is also included in the kit.

It is an object of the present invention to provide a platform for easy and cost-efficient fabrication of bio-microarrays.

It is a feature of the present invention that no modifications to biomolecules for the purpose attaching to the hybrid film are required prior to their immobilization onto the platform for forming a microarray.

It is an advantage of the present invention that the platforms provided for fabricating bio-microarrays are compatible with most commercial printing (spotting) technologies and scanning analysis equipments.

Other advantages, features and objects of the invention will become apparent from the following detailed description when considered in conjunction with the accompanying claims and drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All references mentioned herein are incorporated by reference in their entirety. In case of conflict, the present specification, including definitions, will control.

### BRIEF DESCRIPTION OF THE DRAWINGS

FIG. 1 shows embodiments of procedures for preparing hybrid film-coated slides and microarrays.

FIG. 2 shows the effect of polyelectrolyte thin film thickness on binding capacity (A) and spot size (B). A Cy3-labeled 20-mer oligonucleotide at 25 μM was spotted onto the glass slides coated with different thicknesses of PET. After washing with a solution of 10 mM NaOH and 50 mM Na₂CO₃, the microarray was scanned and analyzed as described in Materials and Methods. Data are mean ±SD of 48 replicates from 3 slides.

FIG. 3 shows the effect of DNA probe concentration and length to the immobilization of DNA probes on multilayered polyelectrolyte thin film (PET) coated slides; the insert graph shows the effects of DNA probes at low concentration. Data are mean ±SD of 48 replicates from 3 slides.

FIG. 4 shows the effects of blocking reagents on microarray performance. 16S-20P and 16S-20M probes were spotted in parallel on 18 of PET coated slides. Before the glass slides were hybridized with the target DNA at 42° C., the slides were passaviated with one of the reagents described in Materials and Methods (for each blocking experiments, three slides were tested). The data shown are mean ±SD of 48 replicates. (A) Comparison of hybridization signal-to-background ratio of 16S-20P probe after treated with different blocking reagents; (B) Effect of blocking reagents to the specificity of the Fₘ/Fₚ.

FIG. 5 compares the binding capacities and hybridization efficiencies on different sides. Dilutions of Cy3-labeled and unlabeled 50-mer probes in printing buffer were immobilized on two separate batches on five types of slides. Slides with Cy3-labeled probes were used to determine immobilization efficiencies after extensive washing, while the slides with unlabeled probes were hybridized with Cy3-labeled target DNA for 16 h. (A) the binding capacities and (B) hybridized amount of target DNA on: 1, PET slide; 2, aldehyde-dextran slide; 3, SuperAldehyde slide; 4, aminopropyltrimethoxylsilane (APTS) slide; and 5, poly-L-lysine (PLL) slide. Values are mean ±SD from 16 spots; (C) Side-by-side comparison of hybridization image on PET, SuperAldehyde and PLL slides.

FIG. 6 compares the background signal and spot size on slides immobilized with 20-mer probes after blocking and hybridization with Cy3 labeled target. Values are mean ±SD from 160 spots.

FIG. 7 shows detection of nucleotide polymorphisms on oligonucleotides microarrays. (A) Layout of hybridization image on PET slide. (B) Comparison of discrimination of nucleotide mismatches on PET, aminopropyltrimethoxylsilane (APTS) and SuperAldehyde slides. Data are mean ±SD of 12 replicates from three slides.

FIG. 8 shows detection of protein-protein interaction on hybrid film-coated glass slides. Slide probed with: 1, mixed Human IgG-Cy3, Fibronectin-Cy3, and Biotin-BSA Cy3; 2, Human IgG-Cy3 and Biotin-BSA Cy3; 3, Human IgG-Cy3; 4, Fibronectin-Cy3; 5, Biotin-BSA Cy3. BSA was used as a negative control on the microarrays.

FIG. 9 shows the dynamic range of a protein microarray.

### DETAILED DESCRIPTION OF THE INVENTION

It is disclosed here that a hybrid 3-D film having a structure of alternating layers of polycationic and polyanionic polymers can be deposited on a bio-microarray substrate (e.g., glass slide) for fabricating bio-microarrays such as polynucleotide or polypeptide microarrays. It is further disclosed that a polyelectrolyte-silica sol-gel film, which is film that contains silica and a polyelectrolyte, can be used for the same purpose. In comparison to the conventional bio-microarrays, bio-microarrays fabricated on the above film-coated substrates have one or more of the advantages of greater polynucleotide or polypeptide binding capacity, greater hybridization efficiency, lower background signal, and more consistent detection spot morphology. The hybrid films disclosed herein are sufficiently rich in electric charges, 3-D porous structures, and potentially hydrogen bond forming groups so that polynucleotides, polypeptides, and other biological matters (e.g., cellular organelles, cells and tissue samples) can be attached or immobilized on these films without any modification for the purpose of introducing active functional groups for attaching to the films (e.g., DNA modified with biotin for attaching to avidin-adsorbed films).

Although one study showed that monosequence DNA probes can be embedded (encapsulated) into a polyelectrolyte thin film that was formed on quartz crystal microbalance (QCM) Au electrode for detecting a target DNA by hybridization, it was not readily predictable whether such a polyelectrolyte film is suitable for fabricating DNA microarrays given the fundamental difference between a QCM biosensor and a DNA microarray. In fact, the study indicated that DNA probes adsorbed directly on the polyelectrolyte film diffused into adjacent areas suggesting that the film may not be suitable for fabricating microarrays. While the diffusion is not a problem for a QCM biosensor because only one species of DNA probes (DNA probes having the same nucleotide sequence or monosequence DNA probes) is attached thereto, it is a problem, however, for a DNA microarray wherein different species of probes are densely spotted at close range and the diffusion of the probes can lead to the loss of detection specificity. What the inventors contributed here is the demonstration that hybrid films of alternating layers of polycationic and polyanionic polymers and polyelectrolyte-silica sol-gel films are suitable for fabricating bio-microarrays (e.g., polynucleotide and polypeptide microarrays) wherein the distance between the centers of two adjacent detection positions or elements on a consecutive hybrid film of the present invention is 1 mm or less, 0.9 mm or less, 0.8 mm or less, 0.7 mm or less, 0.6 mm or less, 0.5 mm or less, 0.4 mm or less 0.3 mm or less, or 0.2 mm or less. For bio-microarrays, a detection position or element is a detection unit wherein the signal(s) therefrom is detected as a whole. The above feature also distinguishes the present invention from some prior art microarrays wherein discrete, but not consecutive, coatings were applied to avoid the probe diffusion problem.

The term bio-microarray is used in the specification and claims to mean a microarray of biological matters. Examples of biological matters that can form a bio-microarray include biomolecules such as polynucleotides and polypeptides, prokaryotic or eukaryotic cells, organelles of prokaryotic or eukaryotic cells, and plant or animal tissue samples. For each type of biological matter, a bio-microarray of the present invention contains at least two distinct species therefrom. For example, for bio-microarrays of monomeric sequence biomolecules, at least two distinct biomolecules that differ by the monomeric sequence are immobilized on different and known positions (locations) on a hybrid film-coated substrate to form at least two detection elements. Each distinct biomolecule of the micrarray may be present as a composition of multiple copies of the molecule on the hybrid film-coated substrate. The number of distinct biomolecules and hence spots or similar microlocations present on the microarray may vary, but is generally at least 2, usually at least 10, and more usually at least 20, where the number of different spots on the micrarray may be as a high as 50, 100, 500, 1,000, 10,000 or higher, depending on the intended use of the microarray. The spots of biomolecules present on the array surface are generally present as a pattern, where the pattern may be in the form of organized rows and columns of spots, e.g. a grid of spots.

The term polynucleotide is used in the specification and claims to mean a molecule that contains a sequence of ribonucleotides or deoxyribonucleotides. Thus, the term covers both DNA and RNA molecules. The sequence of deoxyribonucleotides or ribonucleotides can be short (e.g., oligonucleotide) or long (e.g., PCR amplicon, genomic DNA). In addition to the deoxyribonucleotides or ribonucleotides, a polynucleotide as defined herein may also contain chemically or enzymatically modified nucleotides such as nucleotide analogs.

The term polypeptide is used in the specification and claims to mean a molecule that contains an amino acid sequence. The amino acid sequence can be short (e.g., a short peptide) or long (e.g., a full length protein). A polypeptide as defined herein may also contain chemically or enzymatically modified amino acids.

The term polyelectrolyte, and polyionic polymer are used synonymously in the specification and claims to mean a polymer that has multiple ionic or ionizable groups of the same charge. Similarly, the term polycationic or polyanionic polymer is used to refer to a polyionic polymer with net positive or negative charge.

Any substrates that are known to a skilled artisan as suitable for fabricating bio-microarrays can be used in the platform of present invention. Typically, the solid substrates or the coated surface of the solid substrates are planar in shape. Examples of suitable substrates include but are not limited to those that are made of glass, silica or plastic (e.g., nylon). Preferably, the thickness of a hybrid film of alternating layers of polycationic and polyanionic polymers ranges from 10 nanometer to 10 micrometers, from 20 nanometers to 1 micrometer, or from 50 nanometers to 200 nanometers; the thickness of a hybrid polyelectrolyte-silica sol-gel film ranges from 100 nanometers to 1 millimeter, from 200 nanometers to 500 micrometers, or from 500 nanometers to 200 micrometers.

There are many ways that a film of alternating layers of polyionic polymers of opposite charges can be formed and the present invention is not limited to any particular way of forming the film. For example, polyionic polymers containing a desirable number of ion groups can be deposited onto a charged solid substrate directly. Alternatively, a polymer of no or lower than desirable number of ion groups can be deposited and additional ion groups can then be introduced into the polymer layer. A skilled artisan is familiar with the techniques for depositing a polymer onto a solid substrate (see e.g., references 21 and 22, which are herein incorporated by reference in their entirety) including how to modify a substrate surface for immobilizing the polymer. For example, if the surface of the substrate is not charged or sufficiently charged for attaching a first polyionic polymer layer by electrostatic adsorption, a skilled artisan can readily modify the surface to carry sufficient charge opposite to that of the first polyionic polymer. In a preferred embodiment, each layer of the film is formed by a self-assembly process and the multi-layers are stacked together by the electrostatic attraction between oppositely charged polyelectrolytes (for self-assembly processes, see e.g., reference 23, which is herein incorporated by reference in its entirety). In this embodiment, a solid substrate is exposed to a solution of a first polyionic polymer allowing the formation of a layer of the polymer through the self-assembly process. Next, the substrate covered with the first polymer is exposed to a solution of a second polyionic polymer that is of the opposite charge of the first polymer to allow formation of a layer of the second polymer on top of the layer of the first polymer. This process is repeated with additional polyionic polymers until a desired number of layers are reached. This method has been shown to produce films of high uniformity and of a well-defined (controllable) thickness. The films created with this technique provide a biological friendly, solution-like environment for biological immobilization and are well suited for microarray fabrication. In this preferred embodiment, the film can be formed without any synthesis work and special equipment.

Each polyionic polymer used for forming the hybrid film in the present invention has a net positive or negative charge from multiple ionic or ionizable functional groups of the same charge. Specifically, the functional groups of the same charge are cations or anions or functional groups that can be ionized to cations or anions. Different cations or anions or groups that can be ionized thereto can be represented in a particular polymer. However, for reasons of accessibility and ease of production, it is preferable that multiple ionic groups in a polymer be identical. Preferably, the polymers used in the present invention are soluble. More preferably, the polymers are soluble in an aqueous solution. The aqueous soluble polycationic polymers preferably have multiple cationic charges at pH 2 or above. The aqueous soluble polyanionic polymers preferably have multiple anionic charges at pH 9 or below.

Examples of polyionic polymers suitable for forming the hybrid film in the present invention include brush copolymers and dendrimers. Brush copolymers are copolymers which have a backbone of one composition and bristles of another. These copolymers are also known as comb copolymers. Dendrimers, also known as dendritic polymers or starburst polymers, are polymers which include a core molecule which is sequentially reacted with monomers with three or more reactive groups, such that at each sequential coupling step, the number of reactive groups at the ends of the polymer increases, usually exponentially.

Representative polycationic polymers include natural and unnatural polyamino acids having net positive charge at neutral pH, positively charged polysaccharides, and positively charge synthetic polymers. Representative polycationic polymers also include polyamines and polyamino acids having amine groups on either the polymer backbone or the polymer sidechains such as poly(L-lysine), poly(D-lysine), poly(omithine), poly(arginine), poly(histidine), poly(aminostyrene), polyacrylamide hydrochloride, poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polypropylenimine dendrimers, poly(N,N,N-trimethylaminoacrylate chloride), poly(methylacrylamidopropyltrimethyl ammonium chloride), polydimethyldiallylammonium chloride (PDDA), polyaminoethylene, poly(aminoethyl)ethylene, polyaminoethylstyrene, and N-alkyl derivatives of the foregoing polymers. Examples of aqueous soluble polymers with positive charges (e.g., at pH 6-8) that can be used in the present invention include but are not limited to poly(L-lysine), poly(D-lysine), poly(arginine), polyallylamine hydrochloride (PAAH), polyethyleneimine (PEI), polyacrylamide hydrochloride (PAAM), polypropylenimine, and polyamindoamine starburst polymers.

Suitable polyanionic polymers include but are not limited to natural and synthetic polymers having net negative charge at neutral pH. Examples of these polymers include poly(4-vinylpyridine) hydrochloride (PVP), polystyrenesulfonate (PSS), polyvinylsulfonate (PVS), dextrinsulfate (DTS), poly(glutamic acid), poly(aspartic acid), heparan sulfate, chondroitin sulfate, dermatan sulfate, dextran sulfate, poly(meth)acrylic acid, oxidized cellulose, and analogs of the foregoing polymers.

The polyelectrolyte polymers used in this invention also include these polymers that have similar structures but with part of the cationic groups such as amine or anionic groups such as carboxylic acid groups substituted with other chemical groups such as methyl, ethyl, ethylene glycol, alkylene oxide, poly(ethylene glycol), and poly(alkylene oxide).

Preferably, the polyelectrolyte polymers used to form the hybrid films have molecular weights between 1,000 and 1,000,000, more preferably between 10,000 and 1,000,000, and most preferably between 30,000 and 200,000.

To form a hybrid film on a solid substrate, a suitable solvent must be used to dissolve the polymers in a homogenous solution. Although water is of somewhat an advantage as it can dissolve the polyelectrolyte alone, the solvent used can vary, depending somewhat upon the polymer being adsorbed. Mixed water-miscible solvents, e.g., water-acetone, water-ethanol and water-tetrahydrofuran (THF), can also be used. The optimum concentration of the polymer can be readily determined by those skilled in the art.

One or more salts can also be added into a polyelectrolyte solution to increase the porosity of the hybrid film and thereby facilitating immobilization of a greater amount of biological matters onto the film. Preferably, the salts are inorganic salts at a concentration from 0.01 M to 2 M. Examples of salts that can be added include but are not limited to salts of Mn²⁺, Cu²⁺, Fe²⁺, Na⁺, NH₄^{+, K}⁺, Ni²⁺, and Mg²⁺.

The hybrid films used in the present invention contain at least two polymers that have ionic groups of opposite charges. Thus, the simplest layer sequence is of the ABABAB . . . type in which A represents one layer and B represents the other. However, the functionality of the layers can be selectively increased by using more than two polymers, for example, ABCBABABCB . . . or ABCDCBADCBAD . . . , in which A and C carry the same charge and B and D carry the same charge opposite to that of A and C. The layer sequence is a consequence of the order of exposure used to apply the individual layers. Slides are preferably rinsed between individual applications to remove residual amounts of polymers that have not bonded or have been only loosely adsorbed to the support. The process for applying the layers of film can easily be converted into a continuous procedure by alternately passing the modified substrate through baths containing the polymers with solvents, and baths containing rinsing liquids.

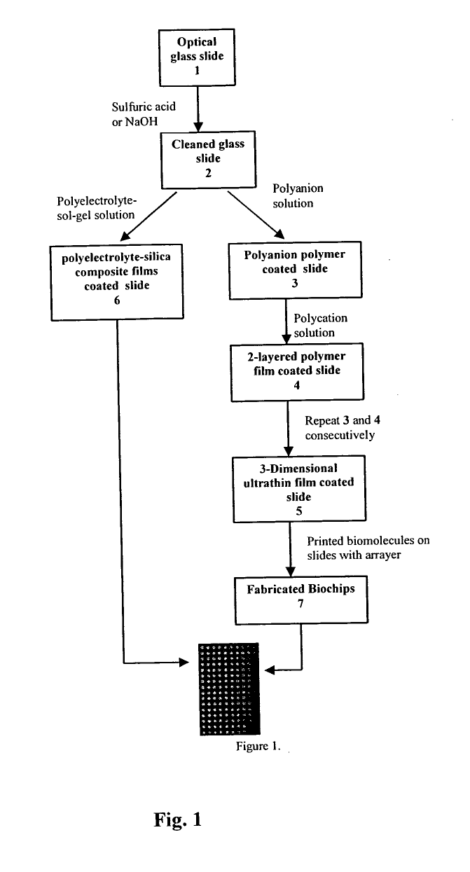

As an example, a protocol for producing a platform and a microarray of the present invention is as follows (FIG. 1): Optical glass slide 1 is cleaned, then the cleaned glass slide 2 is dipped into a polyanionic polymer solution to deposit a polymer layer 3 on the surface, and then dipped into a polycationic polymer solution to deposit a polycationic polymer layer 4. The 3-D hybrid film coated slide 5 is created by repeating five or six alternate adsorptions of every type of polyelectrolyte pair such as PSS/PAAH, PSS/PEI, PVS/PEI, DTS/PAAH, PVS/PDDA, PSS/PDDA, and DTS/PDDA. Sodium polystyrenesulfonate (PSS, MW 70,000, Aldrich Co.) at a concentration of 3 mg/mL, polyallylaminehydrochloride (PAAH, MW 50,000-65,000, Aldrich Co.), polyvinylsulfonate (PVS), polydimethyldiallylammonium chloride (PDDA, Aldrich Co.) at a concentration of 2 mg/mL, dextrinsulfate (DTS) at a concentration of 1.5 mg/mL and branched polyethyleneimine (PEI, MW 70,000) at a concentration of 1.5 mg/mL are dissolved in pure water. The pH of the solutions are adjusted by adding HCl or NaOH. The outermost layer of the slide becomes “negative” or “positive” accordingly. The film is then exposed to a solution with a pH value of about 7.5. The film is also exposed to a temperature of about 110° C. for 30 minutes. Then the coated glass slide is used to fabricate the polynucleotide or polypeptide microarray 7 using standard techniques and devices in the art. In polynucleotide microarrays, the outermost layer of the film is positive; in polypeptide microarrays, the outermost layer of the film is negative.

There are many ways that a solid substrate suitable for fabricating microarrays can be coated with a polyelectrolyte-silica sol-gel film and the present invention is not limited by any particular way that the substrate is coated with the film. Generally speaking, the film-coated substrate can be made by adding a polyelectrolyte into a silica sol-gel solution and dipping a solid substrate into the solution. Alternatively, a spin coating method [24] can be used to deposit the polyelectrolyte doped sol-gel solution onto a surface of a solid substrate (see FIG. 1). A skilled artisan is familiar with the sol-gel techniques and the coating techniques that can be used to coat a substrate with a polyelectrolyte-silica sol-gel film.

The polyelectrolytes that can be used to form a polyelectrolyte-silica sol-gel film are as defined and described above for the hybrid film of alternating layers of polycationic and polyanionic polymers. The polyelectrolyte-silica sol-gel film can be formed with any silica sol-gel material. Examples of the sol-gel materials include but are not limited to those made of aminoalkylsiloxanes, aminocarboxyalkylsiloxanes, carboxyalkylsiloxanes, alkoxaysilanes, and a combination thereof.

Polynucleotide or polypeptide microarrays on polyelectrolyte-silica sol-gel film-coated substrate can be fabricated using standard techniques and devices in the art.

The invention will be more fully understood upon consideration of the following non-limiting examples.

### EXAMPLE 1

### Fabrication of DNA Microarrays on Hybrid Polymeric Ultrathin Film Prepared by Self-Assembly of Polyelectrolyte Multilayers

In this example, we show a novel method for the fabrication of oligonucleotide microarrays with unmodified oligonucleotide probes on hybrid 3-D thin films that are deposited on glass slides by consecutive layer-to-layer adsorption of polyelectrolytes. Unmodified oligonucleotide probes were spotted and immobilized on these multilayered polyelectrolyte thin films (PET) by electrostatic adsorption and entrapment on the porous structure of the PET film. The PET provides higher probe binding capacity, and thus higher hybridization signal than that of the traditional 2-D aminosilane and PLL-coated slides. Immobilized probe densities of 3.4×10¹²/cm² was observed for microarray spots on PET with unmodified 50-mer oligonucleotide probes, which is comparable to the immobilized probe densities of alkyamine-modified 50-mer probes end-tethered on aldehyde functionalized slide. Hybridization efficiency study showed that 90% of immobilized probes on PET film are accessible to target DNA to form duplex format in hybridization. The DNA microarray fabricated on PET film has wider dynamic range (about three orders of magnitude) and lower detection limit (0.5 nM) than the conventional amino- and aldehyde functionalized slides. Oligonucleotide microarrays fabricated on these PET-coated slides also had consistent spot morphology. In addition, discrimination of single nucleotide polymorphism of 16S rRNA genes was achieved with the PET-based oligonucleotide microarrays. The PET microarrays constructed by our self-assembly process is cost-effective, versatile, and well suited for immobilizing many types of biological active molecules.

When the PET films are used for fabrication of cDNA microarrays, ultraviolet or thermal cross-linking of cDNA to PET could be used to further stabilize the arrayed spots, which allows the cDNA on PET to be applied in vigorous denature and washing steps. Moreover, the binding capacity and hybridization sensitivity of the microarray on PET can be further increased by using dendrimeric polymers [14], such as polyamindoamine starburst polymers as starting materials for preparing PET.

Materials and Methods

Reagents: Microscope glass slides (76×26×1 mm) and glass cover slips were obtained from Sigma-Aldrich. Aldehyde modified slides (SuperAldehyde) were purchased from TeleChem International (Sunnyvale, Calif.) and PLL-coated slides were purchased from Cell Associates (Houston, Tex.). Cy3-NHS ester was purchased from Amersham Biosciences Corp. (Piscataway, N.J.). All other chemicals were purchased from Sigma-Aldrich (St.Louis, Mo.).

Oligonucleotides: Oligonucleotides ranging from 11-mer to 50-mer derived from a sequence region of 16S rRNA genes (see Table 1) were synthesized at Michigan State University's Macromolecular Center. Oligonucleotide probes without alkylamino modification were used to fabricate DNA microarrays on PET-, PLL- and aminopropyltrimethoxylsilane (APTS)-coated slides, while oligonucleotides with alkylamino modification at 3′-end were used to fabricate microarrays on aldehyde activated slides. Probes labeled with Cy3 at the 3′-terminal were used to determine the binding capacities of the slides. A Cy3-labeled 50-mer having partial sequence complementary to the 16S probes was used as a target template.

TABLE 1

Probes and target template used. The 5′-terminus alkylamine modified probes were used for aldehyde
functionalized slides and unmodified probes were used for polyelectrolyte multilayer (PET),
aminopropyltrimethoxylsilane (ATPS), and poly-L-lysine (PLL) slides. The mismatched base pair(s) in
the probes are bolded and underlined.

| Length | Name | Sequence (5′ → 3′) | |
|---|---|---|---|
| 11-mer | 16S-11P | G AGG TCT TGC G | (SEQ ID NO:1) |
| | 16S-11P-NH₂ | NH₂₋(C6)-G AGG TCT TGC G | (SEQ ID NO:1) |
| | 16S-11P-Cy3 | G AGG TCT TGC G-Cy3 | (SEQ ID NO:1) |
| 20-mer | 16S-20P | AC GCG AGG TCT TGC GAT CCC | (SEQ ID NO:2) |
| | 16S-20P-Cy3 | AC GCG AGG TCT TGC GAT CCC-Cy3 | (SEQ ID NO:2) |
| | 16S-20P-NH₂ | NH₂₋(C6)-AC GCG AGG TCT TGC GAT CCC | (SEQ ID NO:2) |
| | 16S-20M | AC GCG AGG TAT TGC GAT CCC | (SEQ ID NO:3) |
| 30-mer | 16S-30P | CC AAT CAC GCG AGG TCT TGC GAT CCC CCG C | (SEQ ID NO:4) |
| | 16S-30P NH₂ | NH₂₋(C6)-CC AAT CAC GCG AGG TCT TGC GAT CCC CCG C | (SEQ ID NO:4) |
| | 16S-30P-Cy3 | CC AAT CAC GCG AGG TCT TGC GAT CCC CCG C-Cy3 | (SEQ ID NO:4) |
| 40-mer | 16S-40P | CCGCTCCAATCACGCGAGGTCTTGCGATCCCCCGCTTACC | (SEQ ID NO:5) |
| | 16S-40P-NH₂ | NH₂₋(C6)-C CGC TCC AAT CAC GCG AGG TCT TGC GAT CCC CCG CTT ACC | (SEQ ID NO:5) |
| | 16S-40P-Cy3 | CCGCTCCAATCACGCGAGGTCTTGCGATCCCCCGCTTACC-Cy3 | (SEQ ID NO:5) |
| 50-mer | 16S-50P | ATC GGC CGC TCC AAT CAC GCG AGG TCT TGC GAT CCC CCG CTT ACC CCC TC | (SEQ ID NO:6) |
| | 16S-50P-NH₂ | NH₂₋(C6)-ATC GGC CGC TCC AAT CAC GCG AGG TCT TGC GAT CCC CCG CTT ACC | (SEQ ID NO:6) |
| | | CCC TC-Cy3 | |
| | 16S-50P-Cy3 | ATC GGC CGC TCC AAT CAC GCG AGG TCT TGC GAT CCC CCG CTT ACC CCC TC-Cy3 | (SEQ ID NO:6) |
| 19-mer | 16S-P | ACG CGA GGT CTT GCG ATC C | (SEQ ID NO:7) |
| | 16S-P-NH₂ | NH₂₋(C6)-ACG CGA GGT CTT GCG ATC C | (SEQ ID NO:7) |
| | 16S-M1a | ACG CGA GGT GTT GCG ATC C | (SEQ ID NO:8) |
| | 16S-M1a-NH₂ | NH₂₋(C6)-ACG CGA GGT GTT GCG ATC C | (SEQ ID NO:8) |
| | 16S-M1b | ACG CGA GGT ATT GCG ATC C | (SEQ ID NO:9) |
| | 16S-M1b-NH₂ | NH₂₋(C6)-ACG CGA GGT ATT GCG ATC C | (SEQ ID NO:9) |
| | 16S-M1c | ACG CGA GGT TTT GCG ATC C | (SEQ ID NO:10) |
| | 16S-M1c-NH₂ | NH₂₋(C6)-ACG CGA GGT TTT GCG ATC C | (SEQ ID NO:10) |
| | 16S-M2a | ACG CGA GGC GTT GCG ATC C | (SEQ ID NO:11) |
| | 16S-M2a-NH₂ | NH₂₋(C6)-ACG CGA GGC GTT GCG ATC C | (SEQ ID NO:11) |
| | 16S-M2b | ACG CGA GGG GTT GCG ATC C | (SEQ ID NO:12) |
| | 16S-M2b-NH₂ | NH₂₋(C6)-ACG CGA GGG GTT GCG ATC C | (SEQ ID NO:12) |
| | 16S-M2c | ACG CGA GGA ATT GCG ATC C | (SEQ ID NO:13) |
| | 16S-M2c-NH₂ | NH₂₋(C6)-ACG CGA GGA ATT GCG ATC C | (SEQ ID NO:13) |
| | 16S-M2d | ACG CGA GGC ATT GCG ATC C | (SEQ ID NO:14) |
| | 16S-M2d-NH₂ | NH₂₋(C6)-ACG GGA GGC ATT GCG ATC C | (SEQ ID NO:14) |
| | 16S-M2e | ACG CGA GGG ATT GCG ATC C | (SEQ ID NO:15) |
| | 16S-M2e-NH₂ | NH₂₋(C6)-ACG CGA GGG ATT GCG ATC C | (SEQ ID NO:15) |
| | 16S-M2f | ACG CGA GGA TTT GCG ATC C | (SEQ ID NO:16) |
| | 16S-M2f-NH₂ | NH₂₋(C6)-ACG CGA GGA TTT GCG ATC C | (SEQ ID NO:16) |
| | 16S-M2g | ACG CGA GGC TTT GCG ATC C | (SEQ ID NO:17) |
| | 16S-M2g-NH₂ | NH₂₋(C6)-ACG CGA GGC TTT GCG ATC C | (SEQ ID NO:17) |
| | 16S-M2h | ACG CGA GGG TTT GCG ATC C | (SEQ ID NO:18) |
| | 16S-M2h-NH₂ | NH₂₋(C6)-ACG CGA GGG TTT GCG ATC C | (SEQ ID NO:18) |
| | 16S-M3 | ACG CGA GGC GAT GCG ATC C | (SEQ ID NO:19) |
| | 16S-M3-NH₂ | NH₂₋(C6)-ACG CGA GGC GAT GCG ATC C | (SEQ ID NO:19) |
| | 16S-M4 | ACG CGA GCC GAT GCG ATC C | (SEQ ID NO:20) |
| | 16S-M4-NH₂ | NH₂₋(C6)-ACG CGA GCC GAT GCG ATC C | (SEQ ID NO:20) |
| | 16S-M5 | ACG CGA GCC GAA GCG ATC C | (SEQ ID NO:21) |
| | 16S-M5-NH₂ | NH₂₋(C6)-ACG CGA GCC GAA GCG ATC C | (SEQ ID NO:21) |
| 50-mer | target | GA GGG GGA AAG CGG GGG ATC GCA AGA CCT CGC GTG ATT GGA GCG GCC GAT-Cy3 | (SEQ ID NO:22) |

Slide Preparation: Glass slides were cleaned with hot Piranha solution (1:3 ratio of 30% H₂O₂ and H₂SO₄) and then thoroughly rinsed with distilled water and ethanol. Cleaned slides were immersed into 1 mM of APTS/ethanol solution for 30 min to form an APTS monolayer coating on the glass surface with amino functional groups towards the outside. The APTS-modified glass slides were then immersed in approximately 50 ml of 3 mg/mL polysodium styrenesulfonate solution (PSS; MW 70,000), 0.5 M NaCl at pH about 2.0 for 5 min, followed by washing with distilled water, and air drying. The PSS-coated slide was then exposed to approximately 50 ml of 3 mg/mL polyallylamine hydrochloride solution (PAAH; MW 50,000-65,000), 0.5 M NaCl at pH 8.0 for 5 min. The surface was then washed again with distilled water. This procedure was repeated until the desired number of polyelectrolyte pair layers (PSS/PAAH)ₙ were deposited on the slide with the positively charged PAAH on the outer most layer. The slides were then incubated in pH 7.5, 1 M NaCl solution for 20 minutes and baked in oven at 50° C. for 20 minutes. The positively charged slides were then ready for fabrication of DNA microarrays.

For comparison purposes, a new type of dextran-coated slide with aldehyde active groups was prepared as described elsewhere [25, 26]. Briefly, Dextran (Mw 70 KDa) was oxidized to produce aldehyde groups via standard periodate methods [26]. The APTS-coated slide was treated with 0.02 g/ml aldehyde-dextran solution in 0.2 M sodium phosphate buffer at pH 9.0 for 16 h. The slide was then incubated with 0.1 M sodium borohydride solution to reduce the Schiff bases formed between the glass surface and the dextran chain. The slide was then incubated in 0.1 M sodium periodate solution to produce aldehyde groups. After 2 h of reaction, the activated slide was washed with an excess of distilled water and stored at 4° C.

Microarray fabrication: Oligonucleotide microarrays were fabricated on five types of glass slides with different surface chemistries as summarized in Table 2. The 5′-terminal alkylamine-modified oligonucleotides were attached to the aldehyde and aldehyde-dextran functionalized slides while oligonucleotides without amino modifications, were immobilized on PET, PLL, and APTS slides. Oligonucleotide printing solutions were prepared in a solution of DMSO/H₂O=1/1(for PET, PLL, and APTS slides) or 1×TeleChem spotting solution (for SuperAldehyde and aldehyde-dextran functionalized slides). DNA probe samples were arrayed using a PixSys 5500 robotic printer (Cartesian Technologies, Inc., Irvine, Calif.) in 40% relative humidity. The printed slides were incubated overnight at room temperature. Oligonucleotides that were not bound after spotting were removed by washing the slides twice in a solution of 10 mM NaOH and 50 mM Na₂CO₃ for 2 min each, and in distilled water for 2 min.

TABLE 2
Surface chemistry for probe immobilization

| Slide type^{a} | Functional group on slide | 5′-modification at probes |
|---|---|---|
| PET | long-chain hydrophilic | none |
| | polymer containing | |
| | amine groups and pores | |
| APTS | amine groups | none |
| PLL | amine groups | none |
| SuperAldehyde | aldehyde | alkylamine |
| Aldehyde-Dextran | aldehyde | alkylamine |

^{a}PET = polyelectrolyte multilayer film;
ATPS = aminopropyltrimethoxylsilane;
PLL = poly-L-lysine.

Blocking: To optimize blocking protocols, several physical and chemical blocking methods were tested on the microarrays with 16S-P and 16S-M probes fabricated on PET, PLL and APTS functionalized slides by evaluating the hybridization performance. The blocking protocols were: (1) 0.5% BSA, 0.1% SDS in 100 mM PBS buffer for 30 min; (2) 5× Denhardt's solution (containing 1 mg/ml each of Ficoll, polyvinyl pyrrolidone, and bovine serum albumin) for 30 min; (3) 0.5 mg/ml of sodium poly(styrenesulfonate) (PSS, MW 70,000) in 10 mM sodium acetate buffer at pH 7.0 for 10 min; (4) 0.5 M solution of succinic anhydride in N,N-dimethylformamide (DMF) overnight, the slides were carefully washed with DMF 3 times; (5) 0.5 M of glutaric anhydride (GA) in DMF overnight, then the slides were carefully washed with DMF 3 times; and (6) 100 mM solution of 5-formyl-1,3-benzenedisulfonic acid di sodium salt in 100 mM sodium acetate buffer at pH 7.0 for 1 h at room temperature.

For the oligonucleotide microarrays fabricated on aldehyde and aldehyde-dextran slides, the microarrays were passivated by immersing them in a solution containing 0.25 g Na₂BH₄ dissolved in 75 ml 1×PBS and 25 ml EtOH for 5 min, followed by washing three times in 0.2% SDS for 1 min and then in distilled water for 1 min.

Hybridization: Hybridization was accomplished by initially dissolving the Cy3-labeled complementary target in hybridization buffer containing 3×SSC, 40% formamide, and 0.2% SDS. Next, 10 μl of hybridization solution was deposited on the DNA microarray and a glass cover slip was placed on the slide. Hybridization was carried out for 14 h at the 42° C. Following hybridization, the arrays were washed with 1×SSC, 0.2% SDS and 0.1×SSC, 0.2% SDS for 5 min each and then with 0.1×SSC for 30 sec at ambient temperature prior to being dried by centrifugation at 500 g.

Signal detection and data analysis: The microarrays were scanned at 523 nm using a scanning laser confocal fluorescence (ScanArray 5000 System, Packed Biochip Technologies, Boston, Mass.) microscope at 10 μm resolution. For all microarray experiments, the laser power was 80% and the PMT gain was 70%. The images were processed and analyzed using ImaGene 3.0 (Biodiscovery, Inc., Los Angeles, Calif.). Mean signal intensity of each spot was used for data analysis. The local background signal was subtracted automatically from the hybridization signal of each spot. Statistical analysis was performed using SigmaPlot 5.0 (Jandel Scientific, San Rafael, Calif.) or by Microsoft Excel®.

Quantification of immobilized probe DNA and hybridized target DNA: A standard curve of fluorescent intensity versus the Cy3 concentration was generated by detection of the fluorescent signal of Cy3-NHS spots at different concentrations printed on bare glass slide. Cy3-NHS was diluted with printing buffer using a 2-fold dilution series from 50 μM to 0.00185 μM, and ten replicate spots were printed for each dilution at approximately 1 nL in volume. The fluorescent intensities of the spots were examined and plotted against the Cy3 concentration. To detect the binding capacity of each slide, a series of diluted Cy3-labeled 11- to 50-mer oligonucleotides were arrayed on the slide, and the fluorescent intensities of the spots were quantified after washing, and the amount of attached oligonucleotides were then deduced from the standard curve and converted to binding coverage of DNA (in molecules/cm²). To determine the amount of hybridized DNA, microarrays were prepared using unlabeled probes and then treated for hybridization with Cy3-labeled target. The fluorescent intensities of the spots were quantified and the amount of hybridized target DNA was then deduced from the standard curve. Hybridization efficiencies were calculated as the fraction of hybridized target coverage divided by the immobilized probe coverage.

Definition of discrimination factor Fₘ/Fₚ: To evaluate the specificity of oligonucleotide microarray, the discrimination factor, which indicates the ability to differentiate the nucleotide polymorphisms was calculated by using the ratio of hybridization intensity of mismatched probes (Fₘ) to the signal intensity of perfectly matched probe (Fₚ).

Results

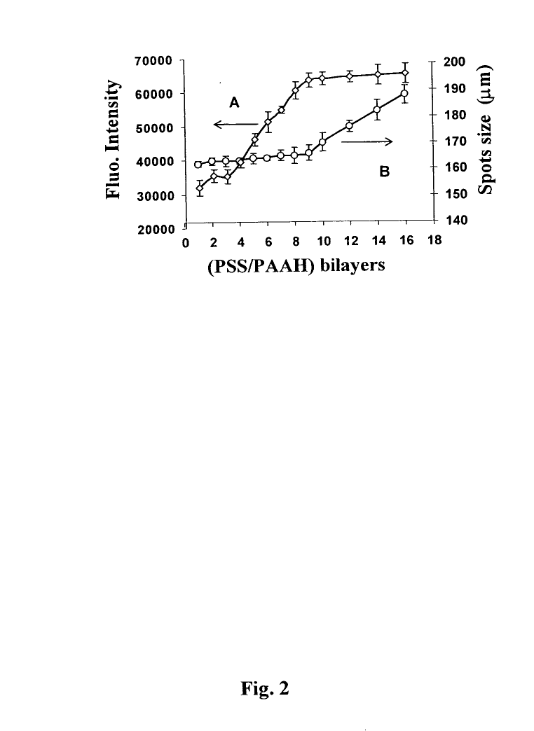

Oligonucleotide immobilization on nanoengineered PET: To optimize the PET film thickness for the construction of DNA microarrays, we spotted the 16S-20P-Cy3 probe onto glass slides that were coated with different bilayers of PSS/PAAH. The effects of film thickness (presented as the number n of bilayers of PSS/PAAH) on probe binding capacity and spot size are shown in FIG. 2. The fluorescent intensity increased with an increase in the number of bilayers and reached a saturation level when the bilayer number (n) was approximately 10 (FIG. 2, A). This correlated with an increase in the number of binding sites (the positively charged amino group and the porous network) on the 3-D PET. The fact that the binding capacity of the PET began to be saturated when the bilayer number was approximately 10 indicated that the DNA probes only penetrate several external polyelectrolyte layers. The use of a contact printing pin may facilitate the direct delivery of DNA probes into the inner layers of the PET. The spot size was constant when the number of bilayers was <9, but increased rapidly when the film thickness was >9 bilayers (FIG. 2, B). This could be because with >9 bilayers of PSS/PAAH, the external polyelectrolyte layers became loose, and caused the spotted probe solution to spread. Thus, the optimized PET thickness was obtained with 9 bilayers of PSS/JPAAH (film thickness 80-100 nm). Glass slides coated with 9 bilayers of PSS/PAAH were therefore used for further study.

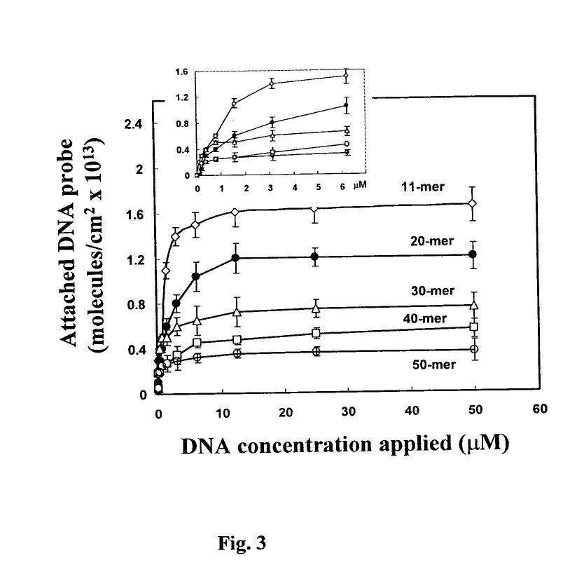

The binding capacities (i.e., the surface coverage) of oligonucleotide probes of different lengths on the (PSS/PAAH)₉ film after washing were further examined by analyzing the signal intensities of microarray spots printed from serial dilutions of each Cy3-labeled oligonucleotide probe. Quantitative data (molecules/cm²) of binding capacities were calibrated from the standard curve. FIG. 3 shows that the coupling efficiency increased as the oligonucleotide concentration increased and reached a plateau at 12.5 μM for all of the oligonucleotide probes. The saturated probe density of the 11- and 50-mer oligonucleotide was 1.7×10¹³ molecules/cm² and 3.4×10¹² molecules/cm², respectively, on the PET. The surface coverage was also related to probe length. The surface coverage decreased with an increase in probe length from 11- to 50-mer, dropping by about an order of magnitude from 1.7×10¹³ to 3.4×10¹² molecules/cm². A decreasing trend in surface coverage with increase of probe length is expected, as it takes fewer large probes to cover a unit area of substrate. The results displayed in FIG. 3 also indicated that synthetic oligonucleotides without modifications as short as 11-mer can be effectively immobilized on the PET.

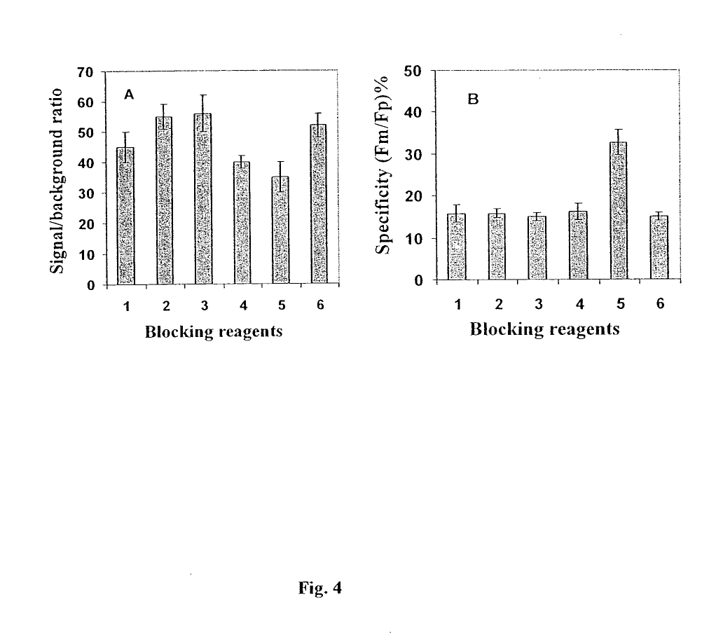

Optimization of blocking protocol for PET slide: The PET films were composed of polyanions comprised of sulfonate groups and polycations comprised of amino groups. The spotted oligonucleotides were bound onto the PET through a combination of non-covalent interaction based on the electrostatic interaction and retention in porous network structures. Although the negatively charged groups on the slide surfaces lead to reduced background signal [14], nonspecific adsorption of target nucleic acids may be caused by the positively charged amino groups and pores of the PET film. We therefore tested several physical and chemical blocking methods on the microarrays fabricated with a perfect match probe (16S-20P) and a probe having a single base-pair mismatch (16S-20M) by evaluating the hybridization performance after blocking. The physical blocking methods cap the unused positively charged groups on the microarray surface by physical adsorption of neutral molecules, while chemical blocking methods convert surface amino groups into negatively charged carboxyl groups or sulfonic group. FIG. 4 shows the array performance obtained from the six blocking experiments. Overall, the physical blocking with 5× Denhardt's solution and the chemical blocking with 10 mM solution of 5-formyl-1,3-benzenedisulfonic acid disodium gave the highest signal-to-noise ratio after hybridization (FIG. 4A). The use of succinic anhydride and glutaric anhydride (GA) as blocking reagents yielded negatively charged surfaces that could not support hybridization (lower hybridization signal) although the background noise of the blocked surface was relatively lower. Moreover, the discrimination ability to identify single nucleotide mismatches (Fₘ/Fₚ) remained consistent except with the use of GA as a blocking reagent for which an unusual loss of specificity was observed (FIG. 4B). This is probably due to the reaction of GA with part of the nucleoside of the DNA probe. Due to its simplicity, 5× Denhardt's solution was therefore used as blocking reagent for PET slides.

Comparison of binding capacities and hybridization efficiencies with other types of slides: PET slides and other four chemically modified glass surfaces were studied for their characteristics relating to the immobilization of oligonucleotides, hybridization efficiency, resulting slide background signal after hybridization, spotting uniformity and specificity to nucleotide polymorphisms. Table 2 lists the slide surface chemistries and the functional modifications of the oligonucleotide probes. These surfaces were selected because they are commonly used in microarray fabrication laboratories.

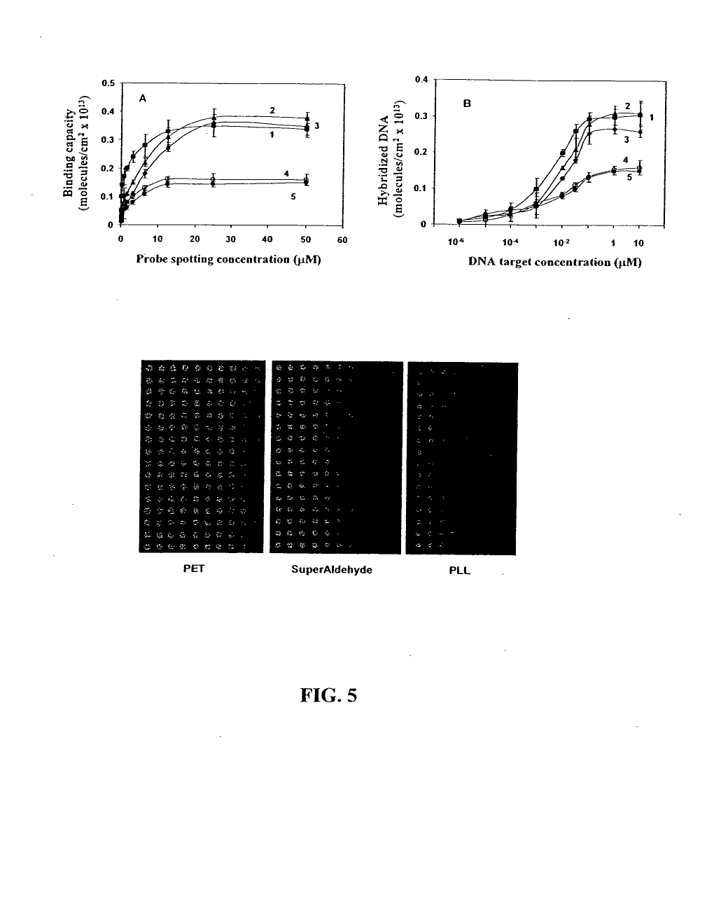

FIGS. 5A and 5B show the comparison of the binding capacities of the 50-mer oligonucleotide probes and their hybridization on the PET slide and four other types of slides. One significant observation was that the probe binding capacity on PET was about 2 fold higher than that of the APTS and PLL slides where the probes were all immobilized on the surface by non-covalent interaction (FIG. 5A). The binding capacity of the unmodified 50-mer probes on PET (3.4×10¹² molecules/cm²) is comparable to the binding capacity on the SuperAldehyde slide (3.6×10¹² molecules/cm²) and the aldehyde-dextran slide (3.8×10¹² molecules/cm²) where the alkylamine modified probes are end-tethered. It is also noteworthy that the PET slides provided high binding capacity even at a low concentration of probe spotting solution. For example, the binding capacity of the unmodified 50-mer probe immobilized on the PET slide was about 2 times greater than the alkylamine modified 50-mer on the SuperAldehyde slide when the concentrations of both the probe spotting solutions were 12.5 μM. Thus, higher concentration probes of 25 μM must be used on aldehyde slides in order to achieve binding saturation.

To compare the hybridization efficiency, unlabeled 50-mer probe at 50 μM was spotted on five types of slides and then hybridized with different concentrations of Cy3-labeled target DNA under the same conditions. The amount of target DNA hybridized on the slides were quantified from the standard curve and plotted against the target concentrations. As shown in FIG. 5B, the amount of target DNA hybridized on the slides with PET was 2.7×10¹²/cm², which is about two-fold higher than the APTS and PLL-coated slides. Unmodified probe immobilized on PET slides is also more accessible to target DNA in hybridization than the alkylamine modified probe immobilized on the aldehyde functionalized slides. The hybridization efficiency of the unmodified 50-mer probe on the PET slide was 90%, whereas it was 70% for the alkylamine modified 50-mer probe on the SuperAldehyde slide. The aldehyde-dextran functionalized slide showed higher binding capacity and hybridization efficiency (approximately 82%) than the SuperAldehyde slide (approximately 70%). It is also noteworthy that the DNA microarray on PET film has wider dynamic range (about three orders of magnitude) and lower detection limit than the other four types of slides. The lowest concentration of target DNA that can be statistically distinguished from background (>backgrounds +3×STD) is 0.5 nM for oligonucleotide microarrays fabricated on PET slide. Displayed in FIG. 5C are the hybridization images obtained on the PET, aldehyde and PLL slides.

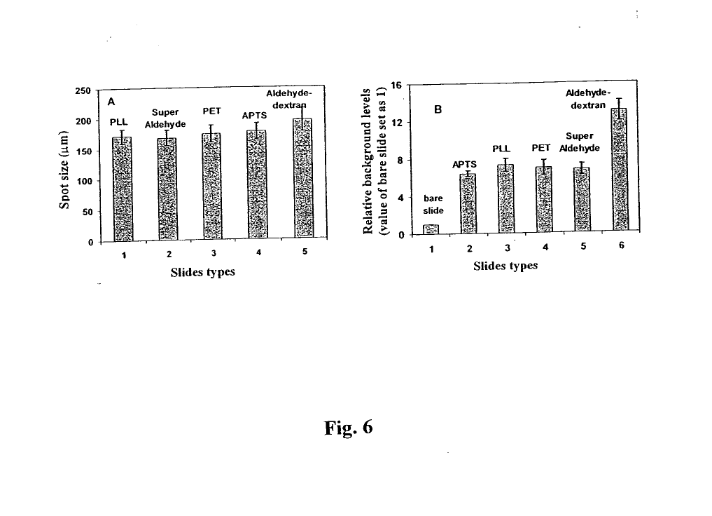

Comparison of background signal and spot size among different slides: FIGS. 6A and 5B compare the spot size and background signal on different slides after hybridization. The size of the spots on the PET slide was 173±10 μm, which was similar to the SuperAldehyde slide (168±10 μm) and the PLL slide (171±10 μm). The background signal of the PET slide was also at the same level as the SuperAldehyde, PLL and APTS slides but with remarkably high and homogenous signal distributions with the individual spots as evidenced by the small standard deviation. This allows for minimized signal deviations of the data, and thus, minimizes experimental errors. The relative high background signal of the aldehyde-dextran slide was probably due to the multi-step synthesis conducted using this type of slide.

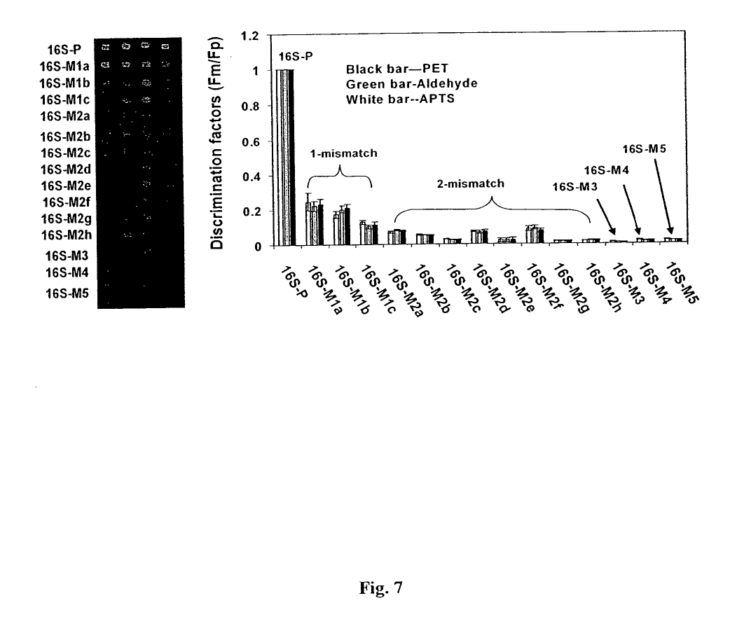

Differentiation of nucleotide polymorphisms: Further studies were performed to evaluate the ability to discriminate single nucleotide polymorphisms using oligonucleotide microarrays fabricated on PET and APTS slides with unmodified probes, and an oligonucleotide microarray aldehyde slides with alkylamine-modified probes. The microarrays were comprised of 15 of 19-mer oligonucleotide probes. Oligonucleotide 16S-P and 16S-P-NH₂ was fully complementary to the part of the Cy3-labeled target present in the hybridization buffer, while oligomers 16S-M1a (and 16S-M1a-NH₂) to 16S-M1c (and 16S-M1c-NH₂) contained a single mismatched nucleotide in the middle with different nucleoside types. Oligonucleotide 16S-M2a to oligonucleotides 16S-M2 h contained two mismatches in the middle and oligonucleotides 16S-M3, 16S-M4, and 16S-M5 contained three, four, and five mismatches, respectively (Table 1). After hybridization under identical conditions, the microarrays on PET, APTS and SuperAldehyde slides were analyzed and the ratios of signal intensities of mismatched probes to perfectly matched probes, Fₘ/Fₚ, were determined. FIG. 7A displayed the image obtained on the PET slide and FIG. 7B shows the Fₘ/Fₚ of each probe determined on the three types of slides. As shown in FIG. 7B, the signal intensities of the oligonucleotides having single mismatched base pairs were discriminated at a signal intensity of 15-25% of the perfectly matched probe, varying with the nucleoside type. The signal intensities of probes with two mismatched nucleosides in the middle were about 5-15% of the perfectly matched probe, whereas oligonucleotide probes containing three, four and five mismatches showed no detectable signal for the target DNA (hybridization signals smaller than about 5% of the perfect matched probe, which is within the standard variation of the statistical analysis), due to the centralized position of three additional mismatches. Overall, the discrimination factor of each probe obtained from the microarrays on the three types of slides was similar. This indicates that the discrimination of nucleotide polymorphisms on an oligonucleotide microarray is independent of the surface chemistry used to immobilize the oligonucleotide probes, although the surface chemistry affects the hybridization signal intensity.

### EXAMPLE 2

### Fabricating Protein Microarrays on Hybrid Film-Coated Glass Slides

This example describes a method of preparing hybrid 3-D film coated glass slides and the fabrication of protein microarrays. The optical glass slide was cleaned with Piranha solution (30% H₂O₂:H₂SO₄/1:3), thoroughly rinsed with distilled water and HPLC purified ethanol, and then dried in air or in a dust-free oven at 50° C. The cleaned slide substrate was then immersed in 50 ml of 1.5 mg/mL PSS aqueous solution with a pH value of approximately 2 for 5 minutes, followed by washing with water and exposure to 50 mL of 3 mg/mL PAAH solution (pH 8.0, adjusted by adding NaOH) for 5 minutes. This surface was then washed with pure water and dried with nitrogen or air. The whole procedure was repeated until 12 polyelectrolyte layers (PSS/PEI)₆ were deposited on the glass surface. Finally, the glass slide was immersed in 50 ml of 1.5 mg/mL PSS aqueous solution with a pH value of approximately 2 for 5 minutes to form the hybrid 3-D film with an outmost layer of negative charge. The hybrid film coated slides were then dried with nitrogen or dust-free ambient air.

Alternatively, the developed hybrid 3-D film coated glass slide was prepared with the following method. Glass microscope slides were cleaned in 2.5 M NaOH for 2 hours, rinsed thoroughly in ultra-pure water, then soaked for 30 minutes in a 3 mg/mL PAAH solution (pH 8.0, adjusted by adding NaOH) for 5 minutes. They were then rinsed in ultra-pure H₂O, and then soaked in a 1.5 mg/mL PSS aqueous solution with a pH value of approximately 2 for 5 minutes. This procedure was repeated until 12 polyelectrolyte layers (PAAH/PSS)₆ were deposited on the glass surface. The slide was rinsed with ultra-pure water and spun dry.

Four antibody/antigen pairs were obtained from a commercial source (anti-human IgG and human IgG, anti-fibronectin and fibronectin, biotinlayted bovine serum albumin and streptavidin). Antibody probe printing solutions were prepared in a dilution series from 0.5 mg/mL to 0.0125 mg/mL in PBS (0.14 M NaCl, 0.003 M KCl, 0.01 M sodium phosphate) and source plates were set up in 384-well plates. The antibody probes were printed at a volume of 500 picoliters per spot, using an arrayer, on the prepared hybrid film coated glass slide. Following printing, the microarrays were incubated for 2 hours at 25° C. at 60% relative humidity. Slides were then washed three times for 5 minutes in a solution of PBS with 0.5% Tween 20 (PBST) to remove any unbound probes. Before immunoassay, the antibody arrays were blocked with 15 μL of 0.5% BSA, and 0.2% Tween 20 PBS solution for 15 minutes. The excess liquid was shaken off. Antibody microarray slides were stored in a solution of 0.5% BSA and 0.2% Tween 20 PBS solution at 4° C. Immunoassays were carried out with a Cy3-labeled antigen solution of 10 μg/mL in 100 mM PBS for 2 hours at room temperature. Without allowing the array to dry, 15 μL of dye-labeled antigen solution at 10 μg/mL in 100 mM PBS was applied to the microarray surface. A 24 mm×30 mm coverslip was placed over the solution. The arrays were sealed in a chamber with an under-layer of PBS to provide humidification, after which they were kept at room temperature for 2 hours. The arrays were dunked briefly in PBS to remove the protein solution and the coverslip, and they were allowed to rock gently in PBS/0.1% Tween 20 solution for 20 minutes. The arrays were then washed twice in PBS for 5-10 minutes each and twice in water for 2-5 minutes each. All washes were at room temperature. After spinning to dryness in a centrifuge, the arrays were scanned with a ScanArray 5000 System.

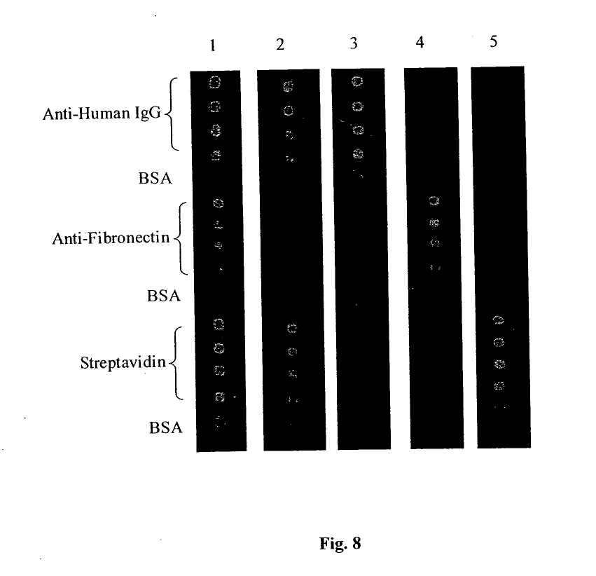

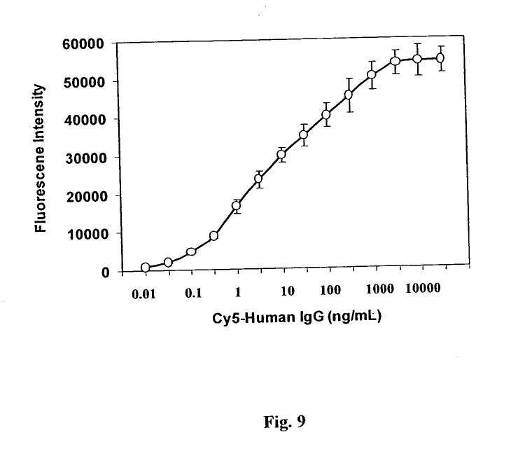

FIG. 8 shows the antibody-antigen interaction on the developed protein microarray. The detection was highly specific and no significant background or nonspecific immunoassaying occurred. To determine the range of sensitivity of this assay, we varied the concentration of both the protein being spotted (anti-human IgG) and the protein in solution (Cy3-human IgG). The signal of the spotted protein began to saturate at concentrations above 0.125 mg/mL. Below this, the fluorescent intensity scaled linearly with decreasing concentrations of anti-human IgG. In the case of solution-phase protein Cy3-human IgG, fluorescent intensity scaled linearly with protein concentration over four orders of magnitude (FIG. 9). Specific binding could be detected using Cy3-human IgG concentrations as low as 100 pg/mL.

Antigen microarrays can also be fabricated on the hybrid 3-D film coated slides. For example, biotin-conjugated BSA printing solution was prepared from concentrations of 0.125 mg/mL to 0.0039 mg/mL in PBS (0.14 M NaCl, 0.03 M KCl, 0.01 M sodium phosphate), and 10 μL of the each solution was transferred to 384-well plates. Protein samples were arrayed with a single pin at a spacing distance of 250 μm in 90, 16×5 patches on the above hybrid film coated slide by using a PixSys 5500 robotic printer (Cartesian Technologies, Inc., Irvine, Calif.) at 60% relative humidity. After printing, the microarrays were incubated for 2 hours at 25° C. in 60% relative humidity. Slides were then washed three times for 5 minutes in a solution of PBS with 0.5% Tween 20 (PBST) to remove any unbound probes. Before immunoassay, the antibody arrays were blocked with 15 μL of 0.5% BSA, 0.2% Tween 20 PBS solution for 15 minutes and the excess liquid was shaken off. Antibody microarrays were stored in a solution of 0.5% BSA, 0.2% Tween 20 PBS solution at 4° C. Immunoassays were carried out with Cy3-labeled, 10 μg/mL streptavidin solution in 100 mM PBS for 2 hours at room temperature. After washing, the slides were scanned using a fluorescence microscope. When the images of protein arrays on the hybrid 3-D film coated slides were compared with those on the commercially available slides(superamine slides, poly-lysine slides, and superaldehyde slides), microarrays on the hybrid 3-D film coated slide had more consistent spot morphologies and lower background signals.

### EXAMPLE 3

### Polyelectrolyte-silica Sol-gel Film Coated Glass Slides For Fabrication of Protein Microarrays

This example describes a method of preparing 3-D polyelectrolyte-silica sol-gel film coated glass slides and the fabrication of protein microarrays. The optical glass slide was cleaned with 10 N NaOH solution, thoroughly rinsed with distilled water and HPLC purified ethanol, and then dried in air or in a dust-free oven at 50° C. Silica sol-gel stock solution was prepared by mixing 4.0 mL TEOS (tetraethxylorthosilicate), 2.0 mL of deionized water and 100 μL HCl. The sol-gel solution was stirred at room temperature for 3 h. The polyelectrolyte-silica composite cocktail solution was achieved by mechanically blending sol-gel stock solution with polystyrenesulfonate (PSS) aqueous solutions. The volume ratio of the appropriate polyelectrolyte solution to the silica sol-gel stock solution was chosen to control the composition of the composite film. The sol-gel derived films were prepared from the freshly formulated polyelectrolyte-containing sol-gel stock solutions by spin-coating the surface of glass slide. A typical procedure for the spin-coating of the films onto the glass slides was as follows: 200 μL of polyelectrolyte-containing sol-gel stock solutions was pipetted onto the surface of the glass slide which was then spun at 3,000 rpm for 30 seconds. The film was then dried under ambient room conditions overnight or longer. Alternatively, the polyelectrolyte-silica sol-gel film can also be deposited on the glass slide surface by dipping the glass slide into the polyelectrolyte-containing sol-gel stock solutions. After washing with pure water and dried in air, the polyelectrolyte-silica sol-gel film coated glass slides are ready for fabrication of protein microarray.

### EXAMPLE 4

### Polyelectrolyte-silica Sol-gel Film Coated Glass Slides For Fabrication of DNA Microarrays

This example describes a method of preparing 3-D polyelectrolyte-silica sol-gel film coated glass slides and the fabrication of DNA microarrays. The optical glass slide was cleaned with 10 N NaOH solution, thoroughly rinsed with distilled water and HPLC purified ethanol, and then dried in air or in a dust-free oven at 50° C. Silica sol-gel stock solution was prepared by mixing 4.0 mL MET (α-methyacryloxypropyltrimethoxysilane), 2.0 mL of deionized water and 100 μL HCl. The sol-gel solution was stirred at room temperature for 3 h. The polyelectrolyte-silica composite cocktail solution was achieved by mechanically blending sol-gel stock solution with polydimethylammonium chloride (PDDA) aqueous solutions. The volume ratio of the appropriate polyelectrolyte solution to the silica sol-gel stock solution was chosen to control the composition of the sol-gel film. The sol-gel derived films were prepared from the freshly formulated polyelectrolyte-containing sol-gel stock solutions by spin-coating the surface of glass slide. A typical procedure for the spin-coating of the films onto the glass slides was as follows: 200 μL of polyelectrolyte-containing sol-gel stock solutions was pipetted onto the surface of the glass slide which was then spun at 3,000 rpm for 30 seconds. The film was then dried under ambient room conditions overnight or longer. Alternatively, the polyelectrolyte-silica sol-gel film can also be deposited on the glass slide surface by dipping the glass slide into the polyelectrolyte-containing sol-gel stock solutions. After washing with pure water and dried in air, the polyelectrolyte-silica sol-gel film coated glass slides are ready for fabrication of DNA microarray.

The present invention is not intended to be limited to the foregoing examples, but encompasses all such modifications and variations as come within the scope of the appended claims.

### REFERENCES

1. Lockhart, D. J. and Winterer, E. A. Nature 2000, 405, 827-836.

2. Lander, E. S. Nat. Genet. Suppl., 1999, 21, 3-4.

3. Fodor, S. P. A.; Read, J. L.; Pirrung, M. C.; Stryer, L.; Lu, A. T.; Solas, D. Science, 1991, 251, 767-773.

4. Hacia, J. G.; Fan, J. B.; Ryder, O.; Jin, L.; Edgemon, K.; Ghandour, G.; Mayer, R. A.; Sun, B.; Hsie, L.; Robbins, C. M.; Brody, L. C.; Wang, D.; Lander, E. S.; Lipshutz, R.; Fodor, S. P. A. Collins, F. S. Nat. Genet. 1999, 22(2) 164-167.

5. Lipshutz, R. J.; Fodor, S. P. A.; Gingeras, T. R.; Lockhart, D. J. Nat. Genet. 1999, 21, 20-24.

6. Rogers, Y. H.; Baucom, P. J.; Huang, Z. J.; Bogdanov, V.; Anderson, S.; Boyce-Jacino, M. T. Anal.Biochem. 1999, 266, 23-30.

7. Dolan, P. L.; Wu, Y.; Ista, L. K.; Metzenberg, R. L.; Nelson, M. A.; Lopez, G. P. Nucleic Acid Res. 2001, 29, el07.

8. Lindroos, K.; Liljedahl, U.; Raitio, M.; Syvanen, A.; Nucleic Acid Res. 2001, 29; e69.

9. Guo, Z.; Guilfoyle, R. A.; Thiel, A. J.; Wang, R.; Smith, L. M. Nucleic Acid Res. 1994, 22, 5456-5465.

10. Lee, P. H.; Sawan, S. P.; Modrusan, Z.; Arnold, L. J.; Reynolds, M. A. Bioconjugate Chem. 2002, 13, 97-103.

11. Podyminogin, M. A.; Lukhtanov, E. A.; Reed, M. W. Nucleic Acids Res. 2002, 29, 5090-5098.

12. Belosludtsev, Y.; Iverson, B.; Lemeshko, S.; Eggers, R.; Wiese, R.; Lee, S.; Powdrill, T.; Hogan, M. Anal. Biochem. 2001, 292, 250-256.

13. Lemeshko, S. V.; Powdrill, T.; Belosludtsev, Y. Y.; Hogan, M. Nucleic Acid Res. 2001, 29, 3051-3058.

14. Benters, R.; Niemeyer, C. M.; Drutschmann, D.; Blohm, D.; Wohrle, D. Nucleic Acids Res., 2002, 30, e10.

15. Zhao, X. D.; Nampalli, S.; Serino, A. J.; Kumar, S. Nucleic Acid Res. 2001, 29, 955-959.

16. Proudnikov, D.; Timofeev, E. and Mirzabekov, A. D. Anal. Biochem. 1998, 159, 34-41.

17. Ermantraut, E.; Wohlfart, K.; Woelfl, S.; Schulz, T. and Koehler, M. In Ehrfeld, W. (ed.), Miroreaction Technology-Proceedings of the First International Conference on Microreaction Technology, Spring Verlag, Heidelberg, 1997, 332-339.

18. Afanassiev, V.; Hanemann, V.; Wölfl, S. Nucleic Acid Res. 2000, 28(12), e66.

19. Kodadek, T. Chem. Biol. 2001, 8, 105-115.

20. Stillman, B. A.; Tonkinson, J. L. Biotechniques 2000, 29, 630-635.

21. Böhmer, M. R. (1996) Formation and Stability of Multilayers of Polyelectrolytes, Langmuir; 12(15); 3675-3681.

22. Zhou, X. C.; Huang, L. Q.; Li, S. F. Y. (2001) Microgravimetric DNA sensor based on quartz crystal microbalance: comparison of oligonucleotide immobilization methods and the application in genetic diagnosis. Biosensors & Bioelectronics, 16, 85-95.

23. Charlier, V., Laschewsky, A., Meyer, B., Wischerhoff, E.; Multilayers by adsorption of functional polyelectrolytes, MACROMOLECULAR SYMPOSIA, 126, 105-121 January, 1998.

24. Manso-Silvan, A., Fuentes-Cobas, L., Martin-Palma, R. J.; BaTiO3 thin films obtained by sol-gel spin coating, SURF COAT TECH 151, 118-121 Mar. 1, 2002.

25. Massia, S. P.; Stark, J. J. Biomed. Mater. Res. 2001, 56, 390-399.

26. Penzol, G.; Armisen, P.; Femandez-Lafuente, R.; Rodes, L. Guisan, J. M. Biotechnol. and Bioeng. 1998, 60, 518-523.

27. MacBeath, G.; Schreiber, S. L. (2000) Printing proteins as microarrays for high-throughput function determination, Science 289, 1760-1763.

1. A bio-microarray comprising:
a platform that comprises a solid substrate and one consecutive hybrid film coating a surface of the solid substrate wherein the film comprises alternating polycationic and polyanionic polymer layers; and
at least two species of polynucleotide or polypeptide molecules attached directly to the hybrid film to form at least two detection elements, wherein the polynucleotide or polypeptide molecules are free of modifications for the purpose of attaching to the film and wherein the distance between the centers of the two detection elements on the consecutive hybrid film is 1 mm or less.

2. The bio-microarray of claim 1, wherein the solid substrate is made of glass, silica, or plastic.

3. The bio-microarray of claim 1, wherein the polycationic polymer is selected from poly(L-lysine), poly(D-lysine), poly(omithine), poly(arginine), poly(histidine), poly(aminostyrene), polyacrylamide hydrochloride, poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polypropylenimine dendrimers, poly(N,N,N-trimethylaminoacrylate chloride), poly(methylacrylamidopropyltrimethyl ammonium chloride), polydimethyldiallylammonium chloride (PDDA), polyaminoethylene, poly(aminoethyl)ethylene, polyaminoethylstyrene, or N-alkyl derivatives of the foregoing polymers.

4. The bio-microarray of claim 1, wherein the polycationic polymer is selected from poly(L-lysine), poly(D-lysine), poly(arginine), polyallylamine hydrochloride (PAAH), polyethyleneimine (PEI), polyacrylamide hydrochloride (PAAM), polypropylenimine, or polyamindoamine starburst polymers.

5. The bio-microarray of claim 1, wherein the polyanionic polymer is selected from poly(4-vinylpyridine) hydrochloride (PVP), polystyrenesulfonate (PSS), polyvinylsulfonate (PVS), dextrinsulfate (DTS), poly(glutamic acid), poly(aspartic acid), heparan sulfate, chondroitin sulfate, dermatan sulfate, dextran sulfate, poly(meth)acrylic acid, oxidized cellulose, and analogs of the foregoing polymers.

6. The bio-microarray of claim 1, wherein the total number of polymer layers is from 3 to 15.

7. The bio-microarray of claim 1, wherein the total number of polymer layers is from 6 to 12.

8. The bio-microarray of claim 1, wherein the total number of polymer layers is from 8 to 10.

9. The bio-microarray of claim 1, wherein the polynucleotide molecules are DNA molecules.

10. The bio-microarray of claim 1, wherein the distance between the centers of the two detection elements is 0.5 mm or less.

11. The bio-microarray of claim 1, wherein the distance between the centers of the two detection elements is 0.3 mm or less.

12. The bio-microarray of claim 1, wherein the distance between the centers of the two detection elements is 0.2 mm or less.

13. A bio-microarray comprising:
a platform that comprises a solid substrate and a hybrid film coating a surface of the solid substrate wherein the hybrid film comprises alternating polycationic and polyanionic polymer layers; and
a biological matter attached to the film wherein the biological matter is selected from a cellular organelle, a cell, or a tissue sample.

14. A method for making a platform for fabricating a bio-microarray, the method comprising the steps of:
(a) providing a solid substrate suitable for fabricating bio-microarrays;
(b) coating a surface of the solid substrate with a layer of a first polyionic polymer;
(c) coating the layer of the first polyionic polymer on the substrate with a layer of a second polyionic polymer wherein the net charge of the second polyionic polymer is opposite to the net charge of the first polyionic polymer;
(d) optionally, repeating one or more of the above coating steps until the substrate is coated with a desirable number of layers of polymers; and at least one of
(e) exposing the polymers coating the substrate to a solution having a pH value of about 4.5 to about 9.5; and
(f) exposing the polymer-coated substrate to an energy source selected from heat, ultraviolet light, or microwave.

15. The method of claim 14, wherein the coating steps involve exposing the substrate to a solution containing a polyionic polymer.

16. The method of claim 15, wherein the polyionic polymer solution is an aqueous solution.

17. The method of claim 15, further comprising the step of adding a salt into the polyionic polymer solution before exposing the solid substrate to the polyionic polymer solution.

18. The method of claim 17 wherein the salt is an inorganic salt.

19. The method of claim 14, wherein the method comprises both (e) and (f).

20. A platform made according to the method of claim 14.

21. A method for fabricating a bio-microarray comprising the steps of:
providing a platform for fabricating a bio-microarray according to claim 14; and
attaching a biological matter to the platform.

22. A bio-microarray fabricated according to the method of claim 21.

23. A kit comprising:
at least one uncoated substrate suitable for fabricating a bio-microarray of claim 1;
a solution of a polycationic polymer suitable for making a hybrid film-coated substrate wherein the hybrid film comprises alternating layers of polycationic and polyanionic polymers;
a solution of a polyanionic polymer suitable for making the hybrid film-coated substrate; and
an instruction for application of the solutions to the substrate to form a hybrid film-coated substrate.

24. A kit for fabricating a bio-microarray of claim 1, the kit comprising:
a hybrid film-coated substrate wherein the hybrid film comprises alternating layers of polycationic and polyanionic polymers; and
a suitable solution for immobilizing polynucleotides or polypeptides on the hybrid film.

25. A platform for fabricating a bio-microarray, the platform comprising a solid substrate and a polyelectrolyte-silica sol-gel film coating a surface of the solid substrate.

26. A bio-microarray comprising the platform of claim 25 and a biological matter attached to the film of the platform.

27. A method for making the platform of claim 25 comprising the steps of:
providing a solid substrate suitable for fabricating bio-microarrays; and
coating a surface of the solid substrate with a polyelectrolyte-silica sol-gel film.

28. A method for fabricating a bio-microarray comprising the steps of:
providing a platform for fabricating a bio-microarray according to claim 25; and
attaching a biological matter to the film of the platform.


SEQUENCE LISTING (22 sequences)

SEQ ID NO: 1 - DNA, 11 bp, Artificial
  Synthetic DNA probe
gaggtcttgcg

SEQ ID NO: 2 - DNA, 20 bp, Artificial
  Synthetic DNA probe
acgcgaggtcttgcgatccc

SEQ ID NO: 3 - DNA, 20 bp, Artificial
  Synthetic DNA probe
acgcgaggtattgcgatccc

SEQ ID NO: 4 - DNA, 30 bp, Artificial
  Synthetic DNA probe
ccaatcacgcgaggtcttgcgatcccccgc

SEQ ID NO: 5 - DNA, 40 bp, Artificial
  Synthetic DNA probe
ccgctccaatcacgcgaggtcttgcgatcccccgcttacc

SEQ ID NO: 6 - DNA, 50 bp, Artificial
  Synthetic DNA probe
atcggccgctccaatcacgcgaggtcttgcgatcccccgcttaccccctc

SEQ ID NO: 7 - DNA, 19 bp, Artificial
  Synthetic DNA probe
acgcgaggtcttgcgatcc

SEQ ID NO: 8 - DNA, 19 bp, Artificial
  Synthetic DNA probe
acgcgaggtgttgcgatcc

SEQ ID NO: 9 - DNA, 19 bp, Artificial
  Synthetic DNA probe
acgcgaggtattgcgatcc

SEQ ID NO: 10 - DNA, 19 bp, Artificial
  Synthetic DNA probe
acgcgaggttttgcgatcc

SEQ ID NO: 11 - DNA, 19 bp, Artificial
  Synthetic DNA probe
acgcgaggcgttgcgatcc

SEQ ID NO: 12 - DNA, 19 bp, Artificial
  Synthetic DNA probe
acgcgaggggttgcgatcc

SEQ ID NO: 13 - DNA, 19 bp, Artificial
  Synthetic DNA probe
acgcgaggaattgcgatcc

SEQ ID NO: 14 - DNA, 19 bp, Artificial
  Synthetic DNA probe
acgcgaggcattgcgatcc

SEQ ID NO: 15 - DNA, 19 bp, Artificial
  Synthetic DNA probe
acgcgagggattgcgatcc

SEQ ID NO: 16 - DNA, 19 bp, Artificial
  Synthetic DNA probe
acgcgaggatttgcgatcc

SEQ ID NO: 17 - DNA, 19 bp, Artificial
  Synthetic DNA probe
acgcgaggctttgcgatcc

SEQ ID NO: 18 - DNA, 19 bp, Artificial
  Synthetic DNA probe
acgcgagggtttgcgatcc

SEQ ID NO: 19 - DNA, 19 bp, Artificial
  Synthetic DNA probe
acgcgaggcgatgcgatcc

SEQ ID NO: 20 - DNA, 19 bp, Artificial
  Synthetic DNA probe
acgcgagccgatgcgatcc

SEQ ID NO: 21 - DNA, 19 bp, Artificial
  Synthetic DNA probe
acgcgagccgaagcgatcc

SEQ ID NO: 22 - DNA, 50 bp, Artificial
  Synthetic DNA probe
gagggggaaagcgggggatcgcaagacctcgcgtgattggagcggccgat